(19) United States
(12) Patent Application Publication
(10) Pub. No.: US 2012/0070443 A1
(43) Pub. Date: Mar. 22, 2012

(54) PDE1 AS A TARGET THERAPEUTIC IN HEART DISEASE

(75) Inventor: Matthew Movsesian, Salt Lake City, UT (US)

(73) Assignee: UNIVERSITY OF UTAH RESEARCH FOUNDATION, Salt Lake City, UT (US)

(21) Appl. No.: 13/132,547

(22) PCT Filed: Dec. 2, 2009
(86) PCT No.: PCT/US2009/066382

§ 371 Date: Nov. 28, 2011

Related U.S. Application Data
(60) Provisional application No. 61/200,688, filed on Dec. 2, 2008.

(51) Int. Cl. A61K 39/395; A61K 31/7088; A61K 38/02; A61P 9/00; A61P 9/04; C07K 16/40; C12Q 1/44; C40B 30/08; C07K 14/00; C07H 21/00; C07H 21/02; A61K 31/713; A61P 9/06

(52) U.S. Cl. 424/158.1; 514/44 A; 514/44 R; 514/16.4; 435/19; 506/11; 530/350; 536/23.1; 536/24.5; 530/389.8

(57) ABSTRACT

Disclosed are compositions and methods related to inhibition of PDE1.

### CROSS-RELATED APPLICATIONS

This application claims the benefit of U.S. provisional application No. 61/200,688, filed Dec. 2, 2008. The aforementioned application is herein incorporated by this reference in its entirety.

### STATEMENT REGARDING FEDERALLY SPONSORED RESEARCH

This invention was made with government support under the Merit Review grant ‘Isoform-selective PDE3-binding peptides’ funded by the United States Department of Veterans Affairs. The government has certain rights in the invention.

### BACKGROUND

Phosphodiesterases are enzymes that catalyze the degradation of cyclic nucleotides cAMP (cyclic adenosine monophosphate) and cGMP (cyclic guanosine monophosphate), which act on several organs of the human body and of other mammals.

cAMP and cGMP are second messengers that regulate numberless cellular functions such as metabolism, contractility, motility and transcription in practically all the types of cells, including those of the cardiovascular system. Phosphodiesterases degrade cAMP and cGMP and are therefore important regulators of cell function [Polson J. B. and Strada S. J., Ann. Rev. Pharmacol. Toxicol., (1996) 36, 403-427].

cAMP is a nucleotide produced from ATP in response to hormonal stimulation of receptors on the cell surface. It is an important molecule in the transmission of intracellular signals. It acts as a signaling molecule, activating protein kinase A. When hydrolyzed by phosphodiesterases, it is converted to AMP. Once formed, cAMP causes intracellular effects, thus being considered an intercellular hormonal mediator.

cGMP is a nucleotide produced from GTP in response to hormonal stimulation of receptors on the cell surface and inside the cell. It is an important molecule in the transmission of intracellular signals. It acts as a signaling molecule, activating protein kinase G. When hydrolyzed by phosphodiesterases, it is converted to GMP. Once formed, cGMP causes intracellular effects, thus being considered an intercellular hormonal mediator.

Many pathologies related to the functioning of the phosphodiesterases are being studied and the inhibition of the known isoforms has been a treatment mechanism for various diseases. Thus, the PDE1-PDE5 inhibitors have been used for the treatment of the erectile-dysfunction problems [Rosen R. C and Kostis J. B., Am. J. Cardiol., (2003) 92, 9M-18M]; in the treatment of asthma and other inflammatory diseases [Torphy T. J., Am. J. Respir. Crit. Care, Med., (1997) 157, 351-370].

What is needed in the art are inhibitors of PDE1 that can be used to treat subjects with heart diseases and disorders, as well as to treat subjects at risk of the same.

### SUMMARY

Disclosed herein is a method of inhibiting cyclic nucleotide phosphodiesterase 1 (‘PDE1’) in the heart muscle of a subject, the method comprising a) identifying a subject who may benefit from PDE1 inhibition; and b) administering to the subject an inhibitor of PDE1.

In one method, cGMP concentration can be increased in any part of the heart muscle of a subject, the method comprising: a) identifying a subject in need thereof; and b) administering to the subject an inhibitor of PDE1, thereby increasing cGMP concentration.

Also disclosed is a method of increasing cAMP concentration in any part of the heart muscle of a subject, the method comprising: a) identifying a subject in need thereof; and b) administering to the subject an inhibitor of PDE1, thereby increasing cAMP concentration.

Further disclosed is a method of increasing both cAMP and cGMP concentration in any part of the heart muscle of a subject, the method comprising: a) identifying a subject in need thereof; and b) administering to the subject an inhibitor of PDE1, thereby increasing both cAMP and cGMP concentration.

Disclosed herein is a method of treating a subject with heart disease, the method comprising: a) screening for a test compound that inhibits PDE1, by i) contacting PDE1 with a test compound; and ii) detecting interaction between PDE1 and the test compound; iii) determining if the test compound inhibits PDE1; and iv) if the results of step iii are positive, selecting the test compound that inhibits PDE1; b) administering the test compound selected in step iv to a subject in need thereof, thereby treating a subject in need thereof.

Also disclosed is a method of screening for a compound that increases cGMP concentration in any part of the heart muscle by inhibiting PDE1, the method comprising: a) contacting PDE1 with a test compound; b) determining the concentration of cGMP in the presence of the test compound and PDE1; wherein increased concentration of cGMP in the presence of the test compound and PDE1 (relative to cGMP concentration in the absence of the test compound) indicates the test compound increases cGMP concentration.

Further disclosed is a method of screening for a compound that increases cAMP concentration in any part of the heart muscle by inhibiting PDE1, the method comprising: a) contacting PDE1 with a test compound; b) determining the concentration of cAMP in the presence of the test compound and PDE1; wherein increased concentration of cAMP in the presence of the test compound and PDE1 (relative to cAMP concentration in the absence of the test compound) indicates the test compound increases cAMP concentration.

Also disclosed is a method of screening for a compound that increases cGMP and cAMP concentration in any part of the heart muscle by inhibiting PDE1, the method comprising: a) contacting PDE1 with a test compound; b) determining the concentration of cGMP and cAMP in the presence of the test compound and PDE1; wherein increased concentration of cGMP and cAMP in the presence of the test compound and PDE1 (relative to cGMP and cAMP concentrations in the absence of the test compound) indicates the test compound increases cGMP and cAMP concentration.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the consequences of inhibition of PDE3 activity in cardiac muscle. Inhibition of the enzyme blocks hydrolysis and inactivation of cAMP. This potentiates cAMP-mediated intracellular signaling, which, in cardiac muscle, is associated with inotropic effects. PDE3 also hydrolyzes and inactivates cGMP, and its inhibition can potentiate cGMP-mediated signaling, but consequences of inhibition of the cGMP-hydrolytic activity of PDE3 in cardiac muscle have not been described.

FIG. 2 shows putative consequences of inhibition of PDE5 activity in cardiac muscle. In mouse hearts, sildenafil, an inhibitor of PDE5, blocks hydrolysis and inactivation of cGMP. This potentiates cGMP-mediated intracellular signaling, which, in cardiac muscle, is associated with cardioprotective and anti-hypertrophic effects. While the researchers originally documenting these effects of sildenafil hypothesized that they resulted from inhibition of PDE5 activity, experimental data described herein now indicate that these effects were due to inhibition of PDE1 activity.

FIG. 3 shows the consequences of PDE1 inhibition in cardiac muscle. Inhibition of the enzyme blocks hydrolysis and inactivation of both cAMP and cGMP, potentiating both cAMP- and cGMP-mediated signaling.

FIG. 4 shows the effects of sildenafil on cGMP-hydrolytic activity in preparations from mouse left ventricles. Measurements were made in the presence and absence of calcium and calmodulin as described in Vandeput et al 2009. All measurements were made at a cGMP concentration of 0.1 μM. ‘Ca²⁺-independent activity’ refers to activity measured in the absence of calcium and calmodulin; this includes PDE5 activity and unstimulated PDE1 activity. ‘Ca²⁺-dependent activity’ refers to activity measured in the presence of calcium and calmodulin minus activity measured in their absence (i.e., minus Ca²⁺-independent activity); this activity is comprised solely of Ca²⁺/calmodulin-stimulated PDE1 activity. The effect of sildenafil on total cGMP-activity is represented by adding the data in the two graphs. The two graphs show that, at a concentration of 10 nM, sildenafil affects cGMP hydrolysis by inhibiting PDE5 but not PDE1. At 1 μM (1000 nM), sildenafil inhibits cGMP hydrolysis by inhibiting both PDE5 and PDE1. Quantitatively, the inhibition attributable to inhibition of PDE1 activity is of much greater magnitude than the inhibition attributable to inhibition of PDE5 activity.

FIG. 5 shows the effects of sildenafil on cGMP-hydrolytic activity in preparations from human left ventricles. Measurements were made in the presence and absence of calcium and calmodulin (‘Ca²⁺/calmodulin’) and in their absence (‘EGTA’, a reference the calcium-chelating agent used) as described in Vandeput et al 2009. All measurements were made at a cGMP concentration of 0.1 μM. At 10 nM sildenafil, at which concentration the compound does not inhibit PDE1 activity, no inhibition of cGMP-hydrolytic activity could be detected. There was, in contrast, significant inhibition at 1 μM (1000 nM) sildenafil, at which concentration the compound inhibits PDE1.

FIG. 6 shows the effects of sildenafil on cAMP-hydrolytic activity in preparations from normal mouse hearts (bar on left side) and mouse hearts that had been infarcted by ligation of the left anterior coronary artery (bar on right side). At 1 μM (1000 nM), sildenafil inhibits cAMP-hydrolytic activity by inhibiting PDE1 activity. Measurements depicted here were made in the absence of calcium and calmodulin; in their presence, the relative inhibition by 1 μM (1000 nM) sildenafil was of greater magnitude.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As used herein, the terms “manage,” “managing” and “management” refer to the beneficial effects that a subject derives from administration of a prophylactic or therapeutic agent, which does not result in a cure of the disease or diseases. In certain embodiments, a subject is administered one or more prophylactic or therapeutic agents to “manage” a disease so as to prevent the progression or worsening of the disease or diseases.

As used herein, the terms “prevent”, “preventing” and “prevention” refer to the methods to avert or avoid a disease or disorder or delay the recurrence or onset of one or more symptoms of a disorder in a subject resulting from the administration of a prophylactic agent.

The term “pharmaceutically acceptable carrier” is intended to include formulation used to stabilize, solubilize and otherwise be mixed with active ingredients to be administered to living animals, including humans. This includes any and all solvents, dispersion media, coatings, antibacterial and antifungal agents, isotonic and absorption delaying agents, and the like, compatible with pharmaceutical administration. The use of such media and agents for pharmaceutically active substances is well known in the art. See e.g., Remington: The Science and Practice of Pharmacy 20th Ed. (Lippincott, Williams & Wilkins 2003). Except insofar as any conventional media or agent is incompatible with the active compound, such use in the compositions is contemplated.

As used herein, the terms “small interfering RNA” (“siRNA”) or “short interfering RNAs”) refer to an RNA (or RNA analog) comprising between about 10-50 nucleotides (or nucleotide analogs) which is capable of directing or mediating RNA interference. As used herein, “shRNA” should be distinguished from siRNA. As described in Hannon et al., “Unlocking the potential of the human genome with RNA interference,” Nature 431, 371-378 (16 Sep. 2004), shRNA involves expressing mimics of miRNAs in the form of short hairpin RNAs (shRNAs) from RNA polymerase II or III promoters. shRNAs typically have stems ranging from 19 to 29 nucleotides in length, and with various degrees of structural similarity to natural miRNAs. Because these triggers are encoded by DNA vectors, they can be delivered to cells in any of the innumerable ways that have been devised for delivery of DNA constructs that allow ectopic mRNA expression. These include standard transient transfection, stable transfection and delivery using viruses ranging from retroviruses to adenoviruses. Each shRNA expression construct gives rise to a single siRNA.

As used herein, the term “in combination” refers to the use of more than one prophylactic and/or therapeutic agent. The use of the term “in combination” does not restrict the order in which prophylactic and/or therapeutic agents are administered to a subject with a disorder. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to a disorder. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

The terms “higher,” “increases,” “elevates,” or “elevation” refer to increases above basal or control level. The terms “low,” “lower,” “reduces,” or “reduction” refer to decreases below or control levels. For example, basal levels are normal in vivo levels prior to, or in the absence of, addition of a PDE1 inhibitor.

The term “test compound” is defined as any compound to be tested for its ability to interact with PDE1.

The term “any part of the heart muscle” is defined as a single area of the heart, such as one ventricle for example, or any cell within the heart, or any intracellular location (site, compartment or microenvironment), or the entire heart.

The terms “control levels” or “control cells” are defined as the standard by which a change is measured, for example, the controls are not subjected to the experiment, but are instead subjected to a defined set of parameters, or the controls are based on pre- or post-treatment levels.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the compound combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “subject” means all mammals including humans. Examples of subjects includes, but are not limited to, humans, cows, dogs, cats, goats, sheep, pigs, and rabbits.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS

1. General

Heart failure, a condition in which the ability of the heart muscle to contract is impaired, is a major cause of morbidity and mortality. In less severe stages of the disease, agents that block the actions of hormones whose levels are increased in heart failure are effective in improving functional capacity and reducing mortality. In more severe stages of the disease, agents that increase the force of contraction of the heart are often needed in addition.

The treatment of advanced heart failure often involves the use of agents that inhibit a family of enzymes know as cyclic nucleotide phosphodiesterases, referred to herein as phosphodiesterases. These enzymes regulate levels of two cyclic nucleotides that function as intracellular ‘second messengers’, cyclic AMP and cyclic GMP (cAMP and cGMP), by hydrolyzing them to their inactive forms AMP and GMP, respectively. Phosphodiesterase inhibitors increase intracellular concentrations of cAMP and cGMP by blocking this process.

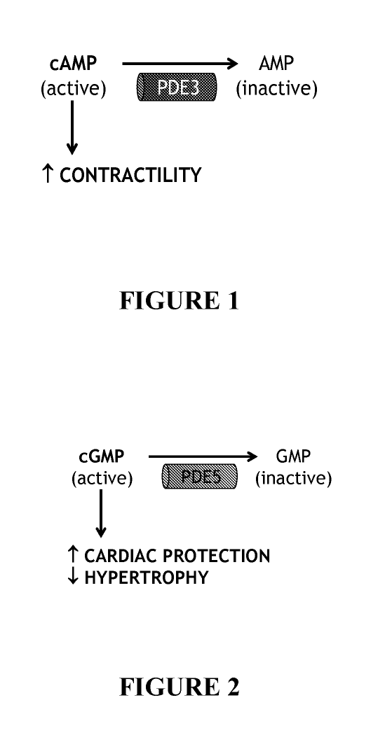

Eleven families of phosphodiesterases have been identified. Drugs that inhibit enzymes in the PDE3 family increase the concentration of cAMP in heart muscle cells. This leads to an increase in the contractility of the heart (FIG. 1). While PDE3 inhibitors are very effective in the short-term, their long-term administration has been associated with a reduction in survival, probably as a consequence of changes in gene expression that lead to increased cell death [Movsesian 2006].

A reason to believe that PDE1 inhibition is preferable to PDE3 inhibition has to do with cGMP-mediated signaling. Studies in animal models have shown cGMP-mediated signaling was stimulated by administration of sildenafil, an inhibitor of the cGMP-selective phosphodiesterase PDE5. Sildenafil was shown to block the development of isoproterenol-induced cardiac hypertrophy in rats, an effect that is accompanied by an increase in survival [Hassan 2005]. Similar observations have been made in mice, where sildenafil blocks and reverses the development of hypertrophy following aortic constriction [Takimoto 2005; Nagayama 2009]. Other benefits have been demonstrated in models of myocardial infarction. Sildenafil reduces infarct size following ischemic injury in rabbit and mouse models [Ockaili 2002; Salloum 2003]. Sildenafil protects against the apoptotic consequences of hypoxia in isolated mouse ventricular myocytes [Das 2005], and anti-apoptotic and survival benefits have been demonstrated in mice whose left anterior descending coronary arteries were ligated [Salloum 2008]. Sildenafil has been shown to have anti-apoptotic effects and to protect against the development of contractile dysfunction in a mouse model of doxorubicin toxicity [Salloum 2008]. The mechanisms involved appear to be dependent upon increases in intracellular cGMP content and the consequent activation of protein kinase G (PKG) [Salloum 2005; Nagayama 2008; Das 2008]. Downstream signaling pathways involving calcineurin/NFAT, phosphoinositide-3 kinase (PI3K)/Akt, and ERK have been implicated, and evidence for the role of ERK phosphorylation and the induction of NO synthases and Bcl-2 in the cardioprotective effects following ischemic injury is especially strong [Takimoto 2005; Das 2008; Das 2009]. In every published report, these beneficial effects of sildenafil have been attributed to inhibition of PDE5, with no mention of the possibility that the beneficial effects of sildenafil might have been attributable to the inhibition of other cyclic nucleotide phosphodiesterases.

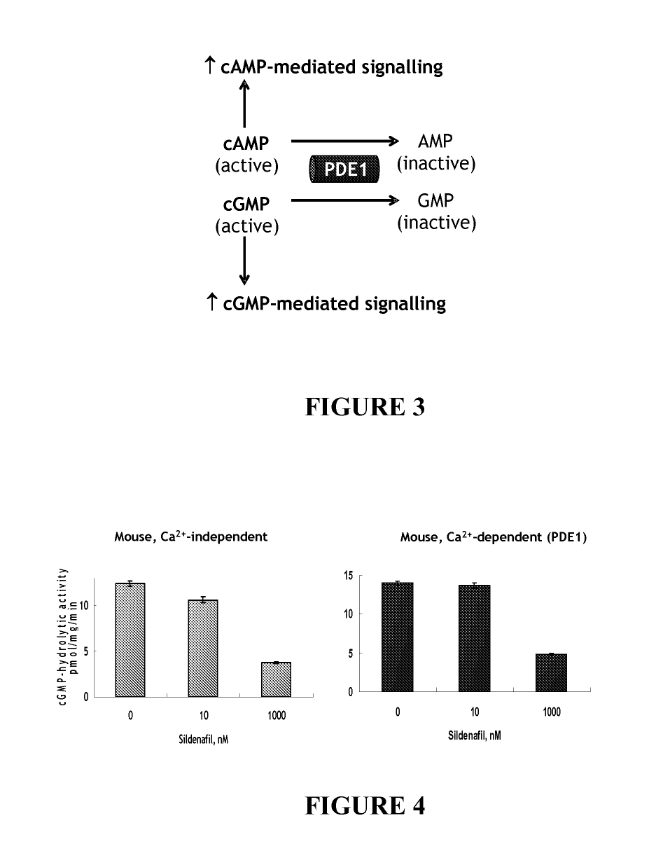

In studies published in 2007, it was shown that another cyclic nucleotide phosphodiesterase, PDE1C1, is present and constitutes the majority of cGMP-hydrolytic activity in normal human myocardium [Vandeput 2007]. This enzyme, whose activity is stimulated in the presence of calcium and calmodulin, hydrolyzes both cAMP and cGMP with similar affinities and kinetic constants Inhibition of PDE1C1 can therefore raise both cAMP and cGMP levels and potentiate both cAMP- and cGMP-mediated signaling (FIG. 3).

In subsequent studies, similar experiments were carried out using diseased human myocardium, with the same result [Vandeput 2009]. In these experiments, the amount of cGMP-hydrolytic activity attributable to PDE5 appeared to be very small. The observation that very little of the cGMP-hydrolytic activity in failing human myocardium was attributable to PDE5 raised the question of whether inhibition of PDE5 was likely to be as beneficial in humans as in mouse models.

It was noted that the concentrations of sildenafil used in many of the animal models of heart disease were in the μM range [Das 2005; Fisher 2005; Pokreisz 2009; Magendran 2007; Takimoto 2007], and it was hypothesized that sildenafil at this concentration could inhibit PDE1 activity as well as PDE5 activity [Vandeput 2009].

The effects of sildenafil on cGMP-hydrolytic activity in preparations from normal (pre-diseased) and diseased mouse left ventricle were then examined [Vandeput 2009]. There was some inhibition of cGMP-hydrolytic activity at 10 nM sildenafil, at which concentration the compound selectively inhibits PDE5 (FIG. 4) Inhibition was greater at 1 μM sildenafil, at which concentration the compound inhibits PDE1 activity (Ca²⁺/calmodulin-stimulated activity) in addition to PDE5 activity. This effect was noted to an even greater degree in pre-diseased than in diseased cardiac muscle. These results led to the novel conclusion that the cardioprotective and other beneficial effects of sildenafil in mouse models were attributable to inhibition of PDE1 activity.

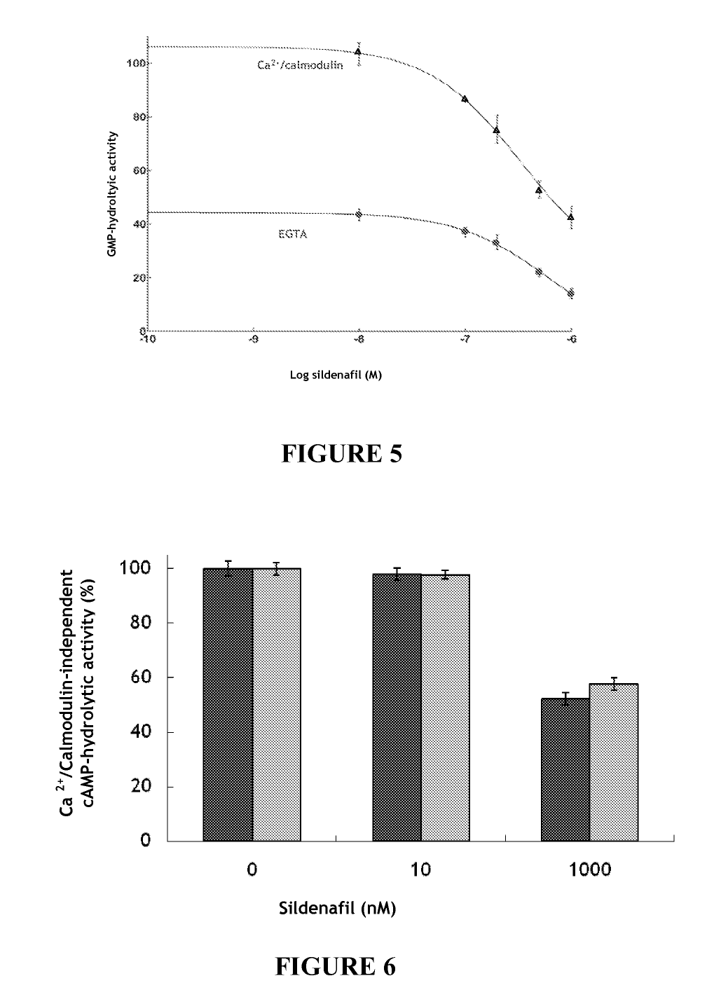

Similar studies were carried out in preparation from human myocardium [Vandeput 2009]. In these studies, no inhibition of cGMP-hydrolytic activity at 10 nM sildenafil was found, whereas inhibition at 1 μM sildenafil was profound (FIG. 5). From this it was inferred that PDE1 inhibition can have cardioprotective and other beneficial effects in human myocardium.

The results shown herein, which include a quantitation of PDE1 and PDE5 activity in preparations from mouse hearts and a quantitation of the effect of sildenafil on these activities, indicate that effects formerly attributed to inhibition of PDE5 activity can be attributed to inhibition of PDE1 activity. As such, they are evidence of cardioprotective and other beneficial effects of inhibiting cardiac PDE1 activity in cardiac disease.

It was also shown that sildenafil, at a concentration of 1 μM, significantly inhibits cAMP-hydrolytic activity in both normal and diseased mouse myocardium (FIG. 6) [Vandeput 2009]. The importance of these new findings can be understood in the context of the ‘compartmentation’ of cyclic nucleotide-mediated (cAMP- and cGMP-mediated) signaling in cardiac myocytes, a term that refers to the fact that cAMP and cGMP levels are regulated specifically and differentially within functionally and spatially distinct compartments of cardiac myocytes. In the case of cAMP-mediated signaling, this compartmentation allows various cAMP-raising extracellular stimuli to elicit different functional responses. β-adrenergic receptor agonists increase cAMP content in cytosolic and microsomal fractions of cardiac myocytes and elicit contractile responses, for example, whereas activation of prostaglandin E1 receptors increases cAMP content only in cytosolic fractions and is without inotropic consequences (Hayes 1982; Hayes 1980). β₁- and β₂-adrenergic receptors are themselves coupled to distinct functional responses, reflecting differences in their spatial distribution in the plasma membrane (Steinberg 1999). β₁-adrenergic receptor stimulation and β₂-adrenergic receptor stimulation elicit qualitatively different cellular responses. In rat cardiac myocytes, β₁-adrenergic receptor agonists increase intracellular Ca²⁺ concentrations during systole, and this increase is associated with an increase in membrane-bound cAMP content. In contrast, the increase in systolic Ca²⁺ concentrations seen with β₂-adrenergic receptor agonists does not correlate with an increase in membrane-bound cAMP content (Xiao 1993; Xiao 1994).

Phosphodiesterases have an important role in this process. In rat cardiac myocytes, β-adrenergic receptor agonists induce increases in intracellular cAMP content that are restricted to discrete spatial microdomains. The addition of a nonspecific phosphodiesterase inhibitor abolishes this specific localization (Zaccolo 2002). PDE3 and PDE4 show distinct localization patterns in these cells, and experiments using PDE3- and PDE4-selective inhibitors indicate a greater role of PDE4 than PDE3 in the increase of cAMP level induced by β-adrenergic stimulation (Mongillo 2004). Experiments using a PDE3-selective inhibitor in rat cardiac myocytes show that PDE3 regulates cAMP signals generated by β₁- and β₂-adrenergic receptor agonists but has no impact on cAMP signals generated by glucagon receptor agonists; PDE4 regulates cAMP signals generated through all three receptors (Rochais 2006). In contrast, in mouse cardiac myocytes, increases in cAMP content induced by β₁-adrenergic receptor agonists are restricted solely by PDE4, while increases induced by β₂-adrenergic receptor agonists are controlled by PDE3 and PDE4 and perhaps other phosphodiesterases (Nikolaev 2006). In another study involving rat hearts, the effects of PDE4 inhibition on β-adrenergic receptor-stimulated cAMP signals seems quantitatively more important than the effects of PDE3 inhibition (Leroy 2006).

cGMP metabolism is also regulated on a compartment-selective basis (Piggott 2006; Castro 2008). Some of this selectivity has been demonstrated through experiments involving the co-expression in cardiac myocytes of two different guanylyl cyclases, particulate guanylyl cyclase (‘pGC’) and soluble guanylyl cyclase (‘sGC’), which show distinct subcellular localizations and are activated by different stimuli. In native tissue, an increase in cGMP-mediated signaling brought about through the stimulation of pGC by natriuretic peptides has been shown to have positive chronotropic and inotropic effects in cardiac myocytes (Beaulieu 1997; Hirose 1998; Wollert 2003). In contrast, stimulation of sGC by NO donors appears to have a negative inotropic effect in cardiac myocytes that is dependent upon the phosphorylation of troponin I by PKG and the consequent decrease in myofilament Ca²⁺ sensitivity (Vila 1999; Wegener 2002; Layland 2002). In another study, performed in perfused rabbit atria, cGMP-mediated inhibition of PDE3 was shown to affect cAMP levels, atrial dynamics and the release of atrial natriuretic peptide from cardiac myocytes differently depending on whether cGMP was produced by pGC or sGC (Wen 2004). Consistent with these studies, spatial confinement of cGMP was recently reported in rat adult ventricular myocytes. In these cells, stimulation of either sGC or pGC leads to the synthesis of cGMP in functionally independent pools having restricted access to individual cGMP-hydrolyzing phosphodiesterases: PDE2 and PDE5 appear to be engaged in degrading cGMP synthesized by sGC, whereas cGMP synthesized by pGC is hydrolyzed solely by PDE2 (Leroy 2006). PDE5 has been shown to be localized to discrete compartments in cardiac myocytes (Takimoto 2004) and to have access only to a fraction of the cGMP produced in these cells (Leroy 2006) showing that it too has a spatially specific role.

These results demonstrate that the increasing intracellular cGMP concentration are highly dependent on the mechanism through which cGMP concentration is increased. For this reasons, there was no way to infer, prior to the findings disclosed herein, that inhibition of the cGMP-hydrolytic activity of PDE1 would have cardioprotective or other beneficial effects. The results disclosed herein are the first to allow this interpretation.

Moreover, since PDE1 hydrolyzes cAMP as well as cGMP, one of skill in the art would have predicted that potential beneficial effects attributable to inhibition of cGMP-hydrolytic activity would be offset by adverse effects attributable to inhibition of cAMP-hydrolytic activity like those seen with inhibition of PDE3, which also hydrolyzes both cAMP and cGMP. The present findings are the first evidence that beneficial effects of PDE1 inhibition are seen despite the inhibition of cAMP-hydrolytic activity.

2. Treatment Methods

Disclosed herein are methods of inhibiting cyclic nucleotide phosphodiesterase 1 (‘PDE1’) in any part of the heart muscle of a subject, the method comprising: a) identifying a subject who may benefit from PDE1 inhibition; and b) administering to the subject an inhibitor of PDE1.

In one method, cGMP concentration can be increased in any part of the heart muscle of a subject, the method comprising: a) identifying a subject in need thereof; and b) administering to the subject an inhibitor of PDE1, thereby increasing cGMP concentration.

Also disclosed is a method of increasing cAMP concentration in any part of the heart muscle of a subject, the method comprising: a) identifying a subject in need thereof; and b) administering to the subject an inhibitor of PDE1, thereby increasing cAMP concentration.

Further disclosed is a method of increasing both cAMP and cGMP concentration in any part of the heart muscle of a subject, the method comprising: a) identifying a subject in need thereof; and b) administering to the subject an inhibitor of PDE1, thereby increasing both cAMP and cGMP concentration.

By “increasing the concentration” is meant increasing over the baseline, or compared to a control, by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or 2, 3, 4, 5, 6, 7, 8, 9, 10, 11, 12, 13, 14, 15, 20, 25, 30, 35, 40, 45, 50, or more fold.

Inhibiting PDE1 can, for example, have a cardioprotective effect. “Cardioprotective effect” refers to actions that prevent or diminish the adverse features or consequences of a variety of pathologic conditions that can affect the heart muscle.

Examples include but are not limited to: acute cardioprotective effects, such as reducing the incidence of sudden death due to arrhythmias or contractile failure in a subject with an acute occlusion of a coronary artery (myocardial infarction); reducing damage occurring during reperfusion of the heart muscle after ischemia (‘hypoxia-reperfusion injury’ or ‘ischemia-reperfusion injury’); reducing the amount of cardiac muscle that is damaged or reducing the severity of damage to the heart muscle caused by an acute coronary artery occlusion (often referred to as ‘reducing infarct size’) Chronic cardioprotective effects include, but are not limited to, reducing pathologic remodeling of the cardiac chambers, including chamber dilation, consequent to an acute coronary artery occlusion; reducing apoptosis in cardiac muscle consequent to an acute coronary artery occlusion; reducing the impairment of contractility of cardiac muscle consequent to an acute coronary occlusion; and reducing long-term mortality in subjects have suffered damage to the heart muscle caused by an acute coronary occlusion.

Acute and/or chronic cardioprotective effects can be desirable in subjects with chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

Inhibiting PDE1 can also have an inotropic effect, increasing the strength of contraction in a failing heart. Acute and chronic inotropic effects may be desirable in acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

Inhibiting PDE1 can also have an anti-arrhythmic effect. This effect can be acute or chronic, and can include effects that are attributable to prevention and/or reduction of injury to the heart muscle. Examples of anti-arrthymic effects include, but are not limited to, reducing the incidence and altering the rates of cardiac arrhythmias (including but not limited to atrial fibrillation, other supraventricular arrhythmias, ventricular tachycardia and ventricular fibrillation) following coronary occlusion.

Inhibiting PDE1 can also have an anti-hypertrophic effect. Anti-hypertrophic effects can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

Inhibiting PDE1 can also have lusitropic effects, improving the relaxation of the heart muscle during diastole. Lusitropic effects can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

Inhibiting PDE1 can also have anti-arrhythmic effects of benefit in the treatment of disorders of the heart rhythm, examples of which include but are not limited to atrial fibrillation, ventricular tachycardia and ventricular fibrillation. These effects, which can include reductions in the incidence and rate of the arrhythmias, can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The subject can also be at an increased risk of developing heart disease. This can include (but is not limited to) individuals with hypertension (systemic or pulmonary), obesity, endocrine disease (including diabetes, thyroid disease, adrenal disease, dysregulation of homocysteine metabolism), iron storage disease, amyolidosis, renal disease, connective tissue disease, infectious diseases, thromboembolic disease, immune diseases, hematologic diseases.

The inhibitor of PDE1 can be used in conjunction with another treatment method. Examples of other treatments that can be used in conjunction with those disclosed herein include, but are not limited to, catecholamines and catecholamine receptor agonists and antagonists (including α- and β-adrenergic receptor agonists and antagonists), vasopressin receptor agonists and antagonists, organic nitrates, activators of soluble and particulate guanylyl cyclases, natriuretic peptides or other components that bind to natriuretic peptide receptors and stimulate or inhibit guanylyl cyclase activity, renin inhibitors, angiotensin-converting enzymes inhibitors, angiotensin receptor antagonists.

Disclosed herein are inhibitors of the catalytic activity of PDE1. Also disclosed is the inhibition of the interactions of PDE1 with PDE1-binding proteins, such as calmodulin. Any suitable source of PDE1 may be employed as an inhibitor target in the present method. The enzyme can be derived, isolated, or recombinantly produced from any source known in the art, including yeast, microbial, and mammalian, that will permit the generation of a suitable product that can generate a detectable reagent or will be biologically active in a suitable assay. A functional fragment or a derivative of PDE1 that still substantially retains its enzymatic activity can also be used.

A compound is an inhibitor of PDE1 expression or biological activity when the compound reduces the expression or activity of PDE1 relative to that observed in the absence of the compound or alters the interactions of PDE1 with PDE1-binding proteins. Inhibitor compounds are those molecules that inhibit or reduce PDE1 functional activity or alter its intracellular distribution. Such inhibition can occur through direct binding of one or more critical binding residues of PDE1 or through indirect interference including steric hindrance, enzymatic alteration of the PDE1, inhibition of transcription or translation, destabilization of mRNA transcripts, impaired export, processing, or localization of PDE1, and the like. Such inhibition can also occur through direct binding of one or more critical binding residues of a PDE1-binding protein or through indirect interference including steric hindrance, enzymatic alteration of the PDE1-binding protein, inhibition of transcription or translation, destabilization of mRNA transcripts, impaired export, processing, or localization of the PDE1-binding protein, and the like. As used herein, the term “inhibitor compound” includes both protein and non-protein moieties. In some embodiments, the inhibitors are small molecules.

In one embodiment, a compound is an inhibitor of PDE1 when the compound reduces the incidence, severity or adverse consequences of heart disease relative to those observed in the absence of the compound, or allows for an increase in concentration of cAMP, cGMP, or both.

Provided herein is a method of increasing or enhancing the chances of survival of a subject with heart disease, comprising administering to a subject in need thereof an effective amount of an inhibitor of PDE1 activity, thereby increasing or enhancing the chances of survival of the subject treated by a certain period of time, for example, by at least 10 days, 1 month, 3 months, 6 months, 1 year, 1.5 years, 2 years, 3 years, 4 years, 5 years, 8 years, or 10 years. The increase in survival of a subject can be defined, for example, as the increase in survival of a preclinical animal model by a certain period of time, for example, by at least 10 days, 1 month, 3 months, 6 months, or 1 year, or at least 2 times, 3 times, 4 times, 5 times, 8 times, or 10 times, more than a control animal model (that has the same type of disease) without the treatment with the inventive method. Optionally, the increase in survival of a mammal can also be defined, for example, as the increase in survival of a subject with heart disease by a certain period of time, for example, by at least 10 days, 1 month, 3 months, 6 months, 1 year, 1.5 years, 2 years, 3 years, 4 years, 5 years, 8 years, or 10 years more than a subject with the same type of heart disease but without the treatment with the inventive method. The control subject may be on a placebo or treated with supportive standard care such as chemical therapy, biologics and/or radiation that do not include the inventive method as a part of the therapy.

Also provided herein is a method of increasing or enhancing the clinical status and perception of well-being of a subject with heart disease, comprising administering to a subject in need thereof an effective amount of an inhibitor of PDE1 activity, thereby increasing or enhancing the chances of survival of the subject treated by a certain period of time,

Examples of PDE1 inhibitors include, but are not limited to, IC86340 (Miller et al., Circ. Res. published online Sep. 24, 2009), 8-methoxymethyl-3-isobutyl-1-methylxanthine; vinpocetine; dioclein (synthesized according to Spearing et al., 1997), and IC295 (compound from ICOS). Several tetracycline guanines that inhibit PDE1 selectively have also been described (Ahn et al, 1997).

The present treatment methods also include a method to increase the efficacy of other agents given for the same disease, comprising administering to a subject in need thereof an effective amount of an inhibitor of PDE1 activity; and, optionally, a pharmaceutically acceptable carrier, thereby increasing the efficacy of the other agent or agents.

a) Functional Nucleic Acids

Functional nucleic acids are nucleic acid molecules that have a specific function, such as binding a target molecule or catalyzing a specific reaction. For example, the functional nucleic acid can inhibit PDE1. Functional nucleic acid molecules can be divided into the following categories, which are not meant to be limiting: antisense molecules, aptamers, ribozymes, triplex forming molecules, and external guide sequences. The functional nucleic acid molecules can act as affectors, inhibitors, modulators, and stimulators of a specific activity possessed by a target molecule.

Functional nucleic acid molecules can interact with any macromolecule, such as DNA, RNA, polypeptides, or carbohydrate chains. Thus, functional nucleic acids can interact with the mRNA of PDE1 or the genomic DNA of PDE1 or they can interact with the polypeptide PDE1 or a domain thereof. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{D}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

b) Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

c) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

d) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as those discussed above that inhibit PDE1, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpes virus amplicon systems are also being used to package pieces of DNA>220 kb and to infect cells that can stably maintain DNA as episomes.

Other useful systems include, for example, replicating and host-restricted non-replicating vaccinia virus vectors.

e) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

g) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

h) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

i) Antibodies

(1) Antibodies Generally

The antibodies disclosed herein can be used as inhibitors of PDE1. The term “antibodies” is used herein in a broad sense and includes both polyclonal and monoclonal antibodies. The antibodies can be tested for their desired activity using the in vitro assays described herein, or by analogous methods, after which their in vivo therapeutic and/or prophylactic activities are tested according to known clinical testing methods. The antibodies disclosed herein can be used to target PDE1.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. Broadly neutralizing anti antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

j) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

k) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semi permeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

l) Therapeutic Uses

Effective dosages and schedules for administering the compositions disclosed herein, such as the PDE1 inhibitors, may be determined empirically, and making such determinations is within the skill in the art. The dosage ranges for the administration of the compositions are those large enough to produce the desired effect in which the symptoms or other pathological aspects of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing ischemic disease or tumor progression, the efficacy of the therapeutic antibody can be assessed in various ways well known to the skilled practitioner.

The disclosed compositions and methods can also be used for example as tools to isolate and test new drug candidates for a variety of heart-related diseases.

3. Screening Methods and Compositions Thereof

Disclosed herein are methods of treating a subject with heart disease, the method comprising: a) screening for a test compound that inhibits PDE1, by i) contacting PDE1 with a test compound; and ii) detecting interaction between PDE1 the test compound; iii) determining if the test compound inhibits PDE1; and iv) if the results of step iii are positive, selecting the test compound that inhibits PDE1; b) administering the test compound selected in step iv to a subject in need thereof, thereby treating a failing or diseased heart.

In the screening methods disclosed herein, the inhibition of PDE1 can be determined by measuring the cGMP and/or cAMP hydrolytic activity of PDE1.

Also disclosed is a method of screening for a compound that increases cGMP concentration in any part of the heart muscle by inhibiting PDE1, the method comprising: a) contacting PDE1 with a test compound; b) determining the concentration of cGMP in the presence of the test compound and PDE1; wherein increased concentration of cGMP in the presence of the test compound and PDE1 (relative to cGMP concentration in the absence of the test compound) indicates the test compound increases cGMP concentration.

Further disclosed is a method of screening for a compound that increases cAMP concentration in any part of the heart muscle by inhibiting PDE1, the method comprising: a) contacting PDE1 with a test compound; b) determining the concentration of cAMP in the presence of the test compound and PDE1; wherein increased concentration of cAMP in the presence of the test compound and PDE1 indicates the test compound increases cAMP concentration.

Also disclosed is a method of screening for a compound that increases cGMP and cAMP concentration in any part of the heart muscle by inhibiting PDE1, the method comprising: a) contacting PDE1 with a test compound; b) determining the concentration of cGMP and cAMP in the presence of the test compound and PDE1; wherein increased concentration of cGMP and cAMP in the presence of the test compound and PDE1 indicates the test compound increases cGMP and cAMP concentration.

Disclosed is a method of identifying a test compound that modulates interaction of PDE1 with one or more further molecules, the method comprising: a) contacting PDE1 with a test compound in the presence of one or more further molecules; and b) determining interaction of PDE1 with one or more further molecules in the presence of the test compound compared to a control; thereby identifying a test compound that modulates interaction of PDE1 with one or more further molecules. In one example, the further molecule can be calmodulin. The test compound can inhibit localization of PDE1 in the cardiac myocytes, for example. Modulation can be inhibition or enhancement.

A variety of different test inhibitory compounds may be used with the methods as provided herein. Test inhibitory compounds can encompass numerous chemical classes. In certain embodiments, they are organic molecules, preferably small organic compounds having a molecular weight of more than 50 and less than about 2,500 daltons. Test inhibitory compounds can comprise functional groups necessary for structural interaction with proteins, particularly hydrogen bonding, and may include at least an amine, carbonyl, hydroxyl or carboxyl group, preferably at least two of the functional chemical groups. The test inhibitory compounds can comprise cyclical carbon or heterocyclic structures and/or aromatic or polyaromatic structures substituted with one or more of the above functional groups. Test inhibitory compounds also include biomolecules like peptides, saccharides, fatty acids, steroids, purines, pyrimidines, derivatives, structural analogs or combinations thereof. Test inhibitory compounds of interest also can include peptide and protein agents, such as antibodies or binding fragments or mimetics thereof, e.g., Fv, F(ab′)₂ and Fab, as described further below.

Test inhibitory compounds also can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The inhibitor can also be prepared and administered as prodrugs. As is known, a pro-drug is a derivative of an active drug, often a relatively simple derivative, whose properties are considerably reduced, compared to those of the drug. The pro-drug is converted to the active drug in the region of the intended action.

a) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

b) Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

c) Compositions Identified by Screening with Disclosed Compositions/Combinatorial Chemistry

(1) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which PDE1 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as PDE1, are also disclosed. Thus, the products produced using the combinatorial or screening approaches that involve the disclosed compositions, such as PDE1 are also considered herein disclosed.

It is understood that the disclosed methods for identifying molecules that inhibit the interactions between, for example, PDE1 and cAMP or cGMP, or both, can be performed using high throughput means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

170. Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein: protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

d) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions are also disclosed. Thus, the products produced using the molecular modeling approaches that involve the disclosed compositions are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

4. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

5. Gene Disruption/Modification

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### C. REFERENCES

1. Das A, Xi L, Kukreja R C. 2005. Phosphodiesterase-5 inhibitor sildenafil preconditions adult cardiac myocytes against necrosis and apoptosis. Essential role of nitric oxide signaling. J Biol Chem 280:12944-55.
2. Fisher P W, Salloum F, Das A, Hyder H, Kukreja R C. 2005. Phosphodiesterase-5 inhibition with sildenafil attenuates cardiomyocyte apoptosis and left ventricular dysfunction in a chronic model of doxorubicin cardiotoxicity. Circulation 111:1601-10.
3. Pokreisz P, Vandenwijngaert S, Bito V, Van den Bergh A, Lenaerts I, Busch C, Marsboom G, Gheysens O, Vermeersch P, Biesmans L, Liu X, Gillijns H, Pellens M, Van Lommel A, Buys E, Schoonjans L, Vanhaecke J, Verbeken E, Sipido K, Herijgers P, Bloch K D, Janssens S P. 2009. Ventricular phosphodiesterase-5 expression is increased in patients with advanced heart failure and contributes to adverse ventricular remodeling after myocardial infarction in mice. Circulation 119:408-16.
4. Nagendran J, Archer S L, Soliman D, Gurtu V, Moudgil R, Haromy A, St Aubin C, Webster L, Rebeyka I M, Ross D B, Light P E, Dyck J R, Michelakis E D. 2007. Phosphodiesterase type 5 is highly expressed in the hypertrophied human right ventricle, and acute inhibition of phosphodiesterase type 5 improves contractility. Circulation 116:238-48.
5. Takimoto E, Belardi D, Tocchetti C G, Vahebi S, Cormaci G, Ketner E A, Moens A L, Champion H C, Kass D A. 2007. Compartmentalization of cardiac beta-adrenergic inotropy modulation by phosphodiesterase type 5. Circulation 115:2159-67.
6. Movsesian M A, Smith C J, Role of cyclic nucleotide phosphodiesterases in heart failure and hypertension, in Cyclic Nucleotide Phosphodiesterases in Health and Disease, Beavo J A, Francis S H, and Houslay M D, Editors. 2006, CRC Press: Boca Raton. p. 485-500.
7. Hassan M A, Ketat A F. 2005. Sildenafil citrate increases myocardial cGMP content in rat heart, decreases its hypertrophic response to isoproterenol and decreases myocardial leak of creatine kinase and troponin T. BMC Pharmacol 5:10.
8. Takimoto E, Champion H C, Li M, Belardi D, Ren S, Rodriguez E R, Bedja D, Gabrielson K L, Wang Y, Kass D A. 2005. Chronic inhibition of cyclic GMP phosphodiesterase 5A prevents and reverses cardiac hypertrophy. Nat Med 11:214-22.
9. Nagayama T, Hsu S, Zhang M, Koitabashi N, Bedja D, Gabrielson K L, Takimoto E, Kass D A. 2009. Sildenafil stops progressive chamber, cellular, and molecular remodeling and improves calcium handling and function in hearts with pre-existing advanced hypertrophy caused by pressure overload. J Am Coll Cardiol 53:207-15.
10. Ockaili R, Salloum F, Hawkins J, Kukreja R C. 2002. Sildenafil (Viagra) induces powerful cardioprotective effect via opening of mitochondrial K(ATP) channels in rabbits. Am J Physiol Heart Circ Physiol 283:H1263-9.
11. Salloum F, Yin C, Xi L, Kukreja R C. 2003. Sildenafil induces delayed preconditioning through inducible nitric oxide synthase-dependent pathway in mouse heart. Circ Res 92:595-7.
12. Salloum F N, Abbate A, Das A, Houser J E, Mudrick C A, Qureshi I Z, Hoke N N, Roy S K, Brown W R, Prabhakar S, Kukreja R C. 2008. Sildenafil (Viagra) attenuates ischemic cardiomyopathy and improves left ventricular function in mice. Am J Physiol Heart Circ Physiol 294:H1398-406.
13. Nagayama T, Zhang M, Hsu S, Takimoto E, Kass D A. 2008. Sustained soluble guanylate cyclase stimulation offsets nitric-oxide synthase inhibition to restore acute cardiac modulation by sildenafil. J Pharmacol Exp Ther 326:380-7.
14. Das A, Xi L, Kukreja R C. 2008. Protein kinase G-dependent cardioprotective mechanism of phosphodiesterase-5 inhibition involves phosphorylation of ERK and GSK3beta. J Biol Chem 283:29572-85.
15. Vandeput F, Wolda S L, Krall J, Hambleton R, Uher L, McCaw K N, Radwanski P B, Florio V, Movsesian M A. 2007. Cyclic nucleotide phosphodiesterase PDE1C1 in human cardiac myocytes. J Biol Chem 282:32749-57.
16. Vandeput F, Krall J, Ockaili R, Salloum F N, Florio V, Corbin J D, Francis S H, Kukreja R C, Movsesian M A. 2009. cGMP-hydrolytic activity and its inhibition by sildenafil in normal and failing human and mouse myocardium. J Pharmacol Exp Ther 330:884-91.
17. Hayes J S, Brunton L L, Mayer S E. 1980. Selective activation of particulate cAMP-dependent protein kinase by isoproterenol and prostaglandin E1. J Biol Chem 255:5113-9.
18. Hayes J S, Bowling N, King K L, Boder G B. 1982. Evidence for selective regulation of the phosphorylation of myocyte proteins by isoproterenol and prostaglandin E1. Biochim Biophys Acta 714:136-42.
19. Steinberg S F. 1999. The molecular basis for distinct beta-adrenergic receptor subtype actions in cardiomyocytes. Circ Res 85:1101-11.
20. Xiao R P, Lakatta E G. 1993. Beta 1-adrenoceptor stimulation and beta 2-adrenoceptor stimulation differ in their effects on contraction, cytosolic Ca²⁺, and Ca²⁺ current in single rat ventricular cells. Circ Res 73:286-300.
21. Xiao R P, Hohl C, Altschuld R, Jones L, Livingston B, Ziman B, Tantini B, Lakatta E G. 1994. Beta 2-adrenergic receptor-stimulated increase in cAMP in rat heart cells is not coupled to changes in Ca²⁺ dynamics, contractility, or phospholamban phosphorylation. J Biol Chem 269:19151-6.
22. Zaccolo M, Pozzan T. 2002. Discrete microdomains with high concentration of cAMP in stimulated rat neonatal cardiac myocytes. Science 295:1711-5.
23. Mongillo M, McSorley T, Evellin S, Sood A, Lissandron V, Terrin A, Huston E, Hannawacker A, Lohse M J, Pozzan T, Houslay M D, Zaccolo M. 2004. Fluorescence resonance energy transfer-based analysis of cAMP dynamics in live neonatal rat cardiac myocytes reveals distinct functions of compartmentalized phosphodiesterases. Circ Res 95:67-75.
24. Rochais F, Abi-Gerges A, Horner K, Lefebvre F, Cooper D M, Conti M, Fischmeister R, Vandecasteele G. 2006. A specific pattern of phosphodiesterases controls the cAMP signals generated by different Gs-coupled receptors in adult rat ventricular myocytes. Circ Res 98:1081-8.
25. Nikolaev V O, Bunemann M, Schmitteckert E, Lohse M J, Engelhardt S. 2006. Cyclic AMP imaging in adult cardiac myocytes reveals far-reaching beta1-adrenergic but locally confined beta2-adrenergic receptor-mediated signaling. Circ Res 99:1084-91.
26. Leroy J, Abi-Gerges A, Nikolaev V O, Richter W, Lechene P, Mazet J L, Conti M, Fischmeister R, Vandecasteele G. 2008. Spatiotemporal Dynamics of {beta}-Adrenergic cAMP Signals and L-Type Ca²⁺ Channel Regulation in Adult Rat Ventricular Myocytes. Role of Phosphodiesterases. Circ Res 11. Castro L R, Verde I, Cooper D M, Fischmeister R. 2006. Cyclic guanosine monophosphate compartmentation in rat cardiac myocytes. Circulation 113:2221-8.
27. Piggott L A, Hassell K A, Berkova Z, Morris A P, Silberbach M, Rich T C. 2006. Natriuretic peptides and nitric oxide stimulate cGMP synthesis in different cellular compartments. J Gen Physiol 128:3-14.
28. Beaulieu P, Cardinal R, Page P, Francoeur F, Tremblay J, Lambert C. 1997. Positive chronotropic and inotropic effects of C-type natriuretic peptide in dogs. Am J Physiol 273:H1933-40.
29. Hirose M, Furukawa Y, Kurogouchi F, Nakajima K, Miyashita Y, Chiba S. 1998. C-type natriuretic peptide increases myocardial contractility and sinus rate mediated by guanylyl cyclase-linked natriuretic peptide receptors in isolated, blood-perfused dog heart preparations. J Pharmacol Exp Ther 286:70-6.
30. Wollert K C, Yurukova S, Kilic A, Begrow F, Fiedler B, Gambaryan S, Walter U, Lohmann S M, Kuhn M. 2003. Increased effects of C-type natriuretic peptide on contractility and calcium regulation in murine hearts overexpressing cyclic GMP-dependent protein kinase I. Br J Pharmacol 140:1227-36.
31. Vila-Petroff M G, Younes A, Egan J, Lakatta E G, Sollott S J. 1999. Activation of distinct cAMP-dependent and cGMP-dependent pathways by nitric oxide in cardiac myocytes. Circ Res 84:1020-31.
32. Wegener J W, Nawrath H, Wolfsgruber W, Kuhbandner S, Werner C, Hofmann F, Feil R. 2002. cGMP-dependent protein kinase I mediates the negative inotropic effect of cGMP in the murine myocardium. Circ Res 90:18-20.
33. Layland J, Li J M, Shah A M. 2002. Role of cyclic GMP-dependent protein kinase in the contractile response to exogenous nitric oxide in rat cardiac myocytes. J Physiol 540:457-67.
34. Wen J F, Cui X, Jin J Y, Kim S M, Kim S Z, Kim S H, Lee H S, Cho K W. 2004. High and low gain switches for regulation of cAMP efflux concentration: distinct roles for particulate GC- and soluble GC-cGMP-PDE3 signaling in rabbit atria. Circ Res 94:936-43.
35. Takimoto E, Champion H C, Belardi D, Moslehi J, Mongillo M, Mergia E, Montrose D C, Isoda T, Aufiero K, Zaccolo M, Dostmann W R, Smith C J, Kass D A. 2004. cGMP Catabolism by PDE5A Regulates Cardiac Adrenergic Stimulation by NOS3-Dependent Mechanism. Circ Res

1. A method of inhibiting cyclic nucleotide phosphodiesterase 1 (‘PDE1’) in any part of the heart muscle of a subject, the method comprising:
a) identifying a subject who may benefit from PDE1 inhibition; and
b) administering to the subject an inhibitor of PDE1.

2. The method of claim 1, wherein the subject has heart disease.

3. The method of claim 1, wherein inhibition of PDE1 has cardioprotective, inotropic, anti-hypertrophic, lusitropic, or anti-arrhythmic effects.

4. The method of claim 3, wherein the cardioprotective effect is acute.

5. The method of claim 3, wherein the cardioprotective effect is chronic.

6-15. (canceled)

16. The method of claim 1, wherein the subject has an increased risk of developing heart disease.

17. The method of claim 1, wherein the inhibitor of PDE1 is an antibody, a small molecule inhibitor, siRNA, shRNA, a polypeptide, a polynucleotide or an antisense polynucleotide.

18. The method of claim 1, wherein the inhibitor is PDE1-selective.

19. The method of claim 1, wherein the inhibitor of PDE1 is used in conjunction with another treatment method.

20. The method of claim 1, wherein the PDE1 inhibitor is contained in a pharmaceutical carrier.

21. A method of increasing cGMP, cAMP, or cGMP and cAMP content or concentration in any part of the heart muscle of a subject, the method comprising:
a) identifying a subject in need thereof; and
b) administering to the subject an inhibitor of PDE1, thereby increasing cGMP, cAMP, or cGMP and cAMP content or concentration.

22-23. (canceled)

24. The method of claim 21, wherein the subject has heart disease.

25. The method of claim 21, wherein inhibition of PDE1 has cardioprotective, inotropic, anti-hypertrophic, lusitropic, or anti-arrhythmic effects.

26. The method of claim 25, wherein the cardioprotective effect is acute.

27. The method of claim 25, wherein the cardioprotective effect is chronic.

28-37. (canceled)

38. The method of claim 21, wherein the subject has an increased risk of developing heart disease.

39. A method of treating a subject with heart disease, the method comprising:
a) screening for a test compound that inhibits catalytic activity of PDE1, by
i) contacting PDE1 with a test compound; and
ii) detecting interaction between PDE1 the test compound;
iii) determining if the test compound inhibits PDE1; and
iv) if the results of step iii are positive, selecting the test compound that inhibits PDE1;
b) administering the test compound selected in step iv to a subject in need thereof, thereby treating a subject with heart disease.

40. The method of claim 39, wherein inhibition of PDE1 is measured by determining the cGMP-hydrolytic activity of PDE1.

41. The method of claim 39, wherein inhibition of PDE1 is measured by determining the cAMP-hydrolytic activity of PDE1.

42. The method of claim 39, wherein inhibition of PDE1 is measured by determining both the cGMP- and cAMP-hydrolytic activity of PDE1.

43. A method of screening for a compound that increases cGMP concentration in any part of the heart muscle by inhibiting PDE1, the method comprising:
a) contacting PDE1 with a test compound;
b) determining the concentration of cGMP in the presence of the test compound and PDE1; wherein increased concentration of cGMP in the presence of the test compound and PDE1 (relative to cGMP concentration in the absence of the test compound) indicates the test compound increases cGMP concentration.

44. A method of screening for a compound that increases cAMP concentration in any part of the heart muscle by inhibiting PDE1, the method comprising:
a) contacting PDE1 with a test compound;
b) determining the concentration of cAMP in the presence of the test compound and PDE1; wherein increased concentration of cAMP in the presence of the test compound and PDE1 indicates the test compound increases cAMP concentration

45. A method of screening for a compound that increases cGMP and cAMP concentration in any part of the heart muscle by inhibiting PDE1, the method comprising:
a) contacting PDE1 with a test compound;
b) determining the concentration of cGMP and cAMP in the presence of the test compound and PDE1; wherein increased concentration of cGMP and cAMP in the presence of the test compound and PDE1 indicates the test compound increases cGMP and cAMP concentration.

46. The method of claim 43, wherein the screening method takes place in vitro.

47. The method of claim 43, wherein the screening method takes place in vivo.

48. The method of claim 43, wherein a plurality of test compounds are contacted with PDE1 in a high throughput assay system.

49. The method of claim 48, wherein the high throughput assay system comprises an immobilized array of PDE1 molecules.

50. A method of identifying a test compound that modulates interaction of PDE1 with one or more further molecules, the method comprising:
a) contacting PDE1 with a test compound in the presence of one or more further molecules; and
b) determining interaction of PDE1 with one or more further molecules in the presence of the test compound, as compared to a control;
thereby identifying a test compound that modulates interaction of PDE1 with one or more further molecules.

51. The method of claim 50, wherein the further molecule is calmodulin.

52. The method of claim 50, wherein the test compound inhibits or alters localization of PDE1 in the cardiac myocyte.

53. The method of claim 50, wherein the test compound inhibits interaction of PDE1 with one or more further molecules.

54. A compound identified by the method of claim 43.
